(19) United States
(12) Patent Application Publication
(10) Pub. No.: US 2014/0249101 A1
(43) Pub. Date: Sep. 4, 2014

(54) METHODS FOR TREATING HEPATITIS C VIRUS INFECTION

(71) Applicant: Gilead Pharmasset LLC, Foster City, CA (US)

(72) Inventors: Xiao Ding, Foster City, CA (US); Robert H. Hyland, Foster City, CA (US); Phillip S. Pang, Foster City, CA (US); William Thomas Symonds, III, San Francisco, CA (US)

(21) Appl. No.: 14/195,751

(22) Filed: Mar. 3, 2014

Related U.S. Application Data
(60) Provisional application No. 61/772,317, filed on Mar. 4, 2013.

(51) Int. Cl. A61K 31/7072; A61K 31/4184; A61K 45/06; A61K 31/7056

(52) U.S. Cl. CPC A61K 31/7072; A61K 31/7056; A61K 31/4184; A61K 45/06
USPC 514/51

(57) ABSTRACT

Disclosed herein is a method of treating a subject infected with hepatitis C virus, said method comprising administering to the subject for a time period an effective amount of sofosbuvir, an effective amount of ribavirin and an effective amount of ledipasvir. In one aspect, the method comprises administering to the subject an interferon-free treatment regimen comprising an effective amount of sofosbuvir, an effective amount of ribavirin and an effective amount of ledipasvir. In a particular aspect, the method is sufficient to produce an undetectable amount of HCV RNA in the subject for at least 12 weeks after the end of the time period.

### CROSS-REFERENCE TO RELATED APPLICATIONS

This application claims the benefit under 35 U.S.C. §119(e) to U.S. Provisional Application No. 61/772,317, filed on Mar. 4, 2013, which is hereby incorporated in its entirety.

### FIELD

Disclosed herein is a method of treating a subject infected with hepatitis C virus, said method comprising administering to the subject for a time period an effective amount of sofosbuvir, an effective amount of ribavirin and an effective amount of ledipasvir. In one aspect, the method comprises administering to the subject an interferon-free treatment regimen comprising an effective amount of sofosbuvir, an effective amount of ribavirin and an effective amount of ledipasvir. In a particular aspect, the method is sufficient to produce an undetectable amount of HCV RNA in the subject for at least 12 weeks or for at least 24 weeks after the end of the time period.

### BACKGROUND

An estimated 170-180 million people are chronically infected with hepatitis C virus (HCV) worldwide (Ghany et al., “Diagnosis, Management, and Treatment of Hepatitis C: An Update,” Hepatology (2009) 49(4):1335-74). In the United States (US), an estimated 3 million people have chronic HCV infection (Dienstag et al., “American Gastroenterological Association Technical Review on the Management of Hepatitis C,” Gastroenterology (2006) 130(1):231-64). Chronic HCV infection can cause chronic liver disease, cirrhosis, liver failure, hepatocellular carcinoma (HCC) and death. As many as 20% of patients with chronic HCV infection are estimated to develop complications, including cirrhosis, end-stage liver disease and hepatocellular carcinoma (Naggie et al., “Hepatitis C Virus Directly Acting Antivirals Current Developments with NS3/4A HCV Serine Protease Inhibitors,” J. Antimicrob. Chemother. (2010) 65(10):2063-9). In 2007 alone, it is estimated that over 15,000 people in the US died from HCV-related complications, and HCV now supersedes human immunodeficiency virus (HIV) as a cause of death in the US (Ly et al., “The Increasing Burden of Mortality from Viral Hepatitis in the United States Between 1999 and 2007,” Ann. Intern. Med. (2012) 156(4):271-8; see also Naggie et al., J. Antimicrob. Chemother. (2010) 65(10):2063-9).

HCV is a single-stranded ribonucleic acid (RNA) virus transmitted primarily through blood or blood product exposure (Centers for Disease Control and Prevention. National Hepatitis C Prevention Strategy—A Comprehensive Strategy for the Prevention and Control of Hepatitis C Virus Infection and its Consequences. 2001). HCV has significant genetic (RNA sequence) variability and is classified on this basis into at least eleven genotypes. Genotype 1 (GT1) is the most common HCV genotype in North America (70%-75%) (Carey, “Tests and Screening Strategies for the Diagnosis of Hepatitis C,” Cleve. Clin. J. Med. (2003) 70(Suppl 4):S7-S13; Anonymous, “National Institutes of Health Consensus Development Conference Statement Management of Hepatitis C 2002 (Jun. 10-12, 2002),” Gastroenterology (2002) 123(6):2082-99) and Europe (69%) (Fattovich et al., “Hepatitis C Virus Genotypes: Distribution and Clinical Significance in Patients with Cirrhosis Type C Seen at Tertiary Referral Centres in Europe,” J. Viral Hepat. (2001) 8(3):206-16).

The initially-approved standard of care (SOC) for the treatment of chronic HCV infection was therapy with pegylated interferon (PEG) either alone or in combination with ribavirin (RBV). The primary goal of treatment for chronic hepatitis C is a sustained virologic response (SVR), which refers to an undetectable level of serum HCV RNA maintained for a period of time post-treatment. The overall response to PEG+RBV combination therapy among patients infected with HCV is only about 50%, and SVR rates are <50% for patients infected with GT1 HCV treated with a prolonged duration (48-72 weeks) of PEG+RBV therapy. (Naggie et al., J. Antimicrob. Chemother. (2010) 65:2063-2069).

A number of potential molecular targets for drug development of direct acting antivirals as anti-HCV therapeutics have now been identified including, but not limited to, the NS2-NS3 autoprotease, the N3 protease, the N3 helicase, and the NS5B polymerase. The RNA-dependent RNA polymerase is essential for replication of the single-stranded, positive sense, RNA genome, and this enzyme has elicited significant interest among medicinal chemists. Another auxiliary protein of HCV is referred to as NS5A. The NS5A nonstructural protein is a phosphoprotein, with no apparent enzymatic activity; however it acts as a multifunctional regulator of cellular pathways, including host cell growth, immunity and innate immunity, and virus replication. (Appel et al., J. Virol. (2005) 79: 3187-3194; Evans et al., Proc. Natl. Acad. Sci. USA (2004) 101: 13038-13043; Gale et al., Nature (2005) 436: 939-945; Gale et al., Virology (1997) 230: 217-227; Ghosh et al., J. Gen. Virol. (1999) 80(Pt 5): 1179-1183; Neddermann et al., J. Virol. (1999) 73: 9984-9991; Polyak et al., Hepatology (1999) 29: 1262-1271; Shimakami et al., J. Virol. (2004) 78: 2738-2748; Shirota et al., J. Biol. Chem. (2002) 277: 11149-11155; and Tan et al., Proc. Natl. Acad. Sci. U.S.A. (1999) 96: 5533-5538.)

NS5A is associated with host cell membranes through its N-terminal amphipathic helix, where it is a part of the replication complex. (Elazar et al., J. Virol. (2004) 78: 11393-11400 and Penin et al., J. Biol. Chem. (2004) 279: 40835-40843.) Recent studies suggest that NS5A is organized into three domains: the first 213 amino acids in the N-terminal domain constitutes domain I and contains a zinc binding motif suggesting that the protein is a zinc metalloprotein and domains II and III are in the C-terminal region of the protein. (Tellinghuisen et al., J. Biol. Chem. (2004) 279: 48576-48587 and Tellinghuisen et al., Nature (2005) 435: 374-379.) NS5A exists in two phosphorylated forms: a basal form of 56 kD and a hyperphosphorylated form of 58 kD. The protein is phosphorylated at specific sites, primarily on serine residue within domains II and III, by host cell kinases. (Ide et al., Gene (1997) 201: 151-158; Kaneko et al., Biochem. Biophys. Res. Commun. (1994) 205: 320-326; Katze et al., Virology (2000) 278: 501-513; Reed et al., J. Biol. Chem. (1999) 274: 28011-28018; Reed et al., J. Virol. (1997) 71: 7187-7197; and Tanji et al., J. Virol. (1995) 69: 3980-3986.)

Peginterferon alfa-2a (PEG-IFN-α-2a or peginterferon α-2a), marketed under the trademark PEGASYS® (Hoffmann-La Roche), is an antiviral administered by subcutaneous injection indicated for, among other things, treatment of chronic hepatitis C(CHC) when administered alone or in combination with ribavirin. PEGASYS® is indicated for the treatment of CHC in patients with compensated liver disease not previously treated with interferon alpha, in patients with histological evidence of cirrhosis and compensated liver disease, and in adults with CHC/HIV co-infection. Combination therapy using PEG-IFN-α-2a and ribavirin is recommended unless the patient has contraindication to or significant intolerance to ribavirin.

Peginterferon alfa-2b (PEG-IFN-α-2b or peginterferon α-2b), marketed under the trademark PEGINTRON® (Merck Sharp & Dohme Corp.), is also administered by subcutaneous injection and is indicated for use alone or in combination with ribavirin to treat CHC in patients with compensated liver disease. Like PEG-IFN-α-2a, PEG-IFN-α-2b has undesirable side effects.

Ribavirin (RBV), marketed under the trademark COPEGUS® (Hoffmann-La Roche), is a nucleoside analogue indicated for the treatment of CHC virus infection in combination with peginterferon in patients 5 years of age and older with compensated liver disease not previously treated with peginterferon, and in adult CHC patients co-infected with HIV. Ribavirin alone is not approved for the treatment of CHC. (COPEGUS® FDA-approved label, revised August 2011.) Clinical trials have shown that ribavirin alone can normalize alanine aminotransferase (ALT) levels transiently during the course of treatment in some patients with CHC infections. However, these studies have reported that ribavirin alone did not reduce HCV RNA levels during or after therapy and did not produce any sustained virologic response. (Di Bisceglie et al., Ann. Intern. Med. (1995) 123(12): 897-903; Dusheiko et al., J. Hepatology (1996) 25: 591-598; Bodenheimer, Jr., et al., Hepatology (1997) 26(2): 473-477.) One clinical study reported observing a decrease in HCV RNA from treatment with ribavirin monotherapy (1.0 to 1.2 g daily for 24 weeks); however, the observed HCV RNA decrease was transient and no patient receiving ribavirin monotherapy cleared HCV RNA. (Pawlotsky et al., Gastroenterology (2004) 126: 703-714.)

Treatment of CHC using peginterferon alone or in combination with ribavirin has several disadvantages. First and foremost, this therapy is not effective for many patients. For instance, certain Phase 3 clinical trials using the combination of peginterferon and ribavirin reported SVR rates of 54 to 63%, but additional studies show that the SVR rates may be much lower in certain populations. (Feurstadt et al., Hepatology (2010) 51(4): 1137-1143.) Second, use of peginterferon and ribavirin is associated with certain adverse events. For instance, the boxed warning on the PEGASYS® label states that use of peginterferon may cause or aggravate fatal or life-threatening neuropsychiatric, autoimmune, ischemic, and infectious disorders. (PEGASYS® (peginterferon alfa-2a) FDA-approved label, revised September 2011.) Additionally, the boxed warning on the COPEGUS® label states that ribavirin adverse effects may include hemolytic anemia and that significant “teratogenic and embryocidal effects have been demonstrated in all animal species exposed to ribavirin.” (COPEGUS® (ribavirin) FDA-approved label, revised August 2011.) Finally, the peginterferon/ribavirin treatment protocol is quite expensive. Given these disadvantages, there has been a recognized need to develop new anti-HCV drug substances and treatment regimens.

Two additional FDA-approved drug products for the treatment of genotype 1 CHC, are boceprevir and telaprevir, both of which are HCV NS3/4 protease inhibitors. Boceprevir, marketed under the trademark VICTRELIS® (Merck Sharp & Dohme Corp), is indicated for the treatment of genotype 1 CHC infection, in combination with interferon and ribavirin, in adult patients (≧18 years of age) with compensated liver disease, including cirrhosis, who are previously untreated or who have failed previous interferon and ribavirin therapy. Telaprevir, marketed under the trademark INCIVEK® (Vertex Pharmaceuticals Inc), is indicated, in combination with interferon and ribavirin, for the treatment of genotype 1 CHC in adult patients with compensated liver disease, including cirrhosis, who are treatment-naïve or who have been previously treated with interferon-based treatment, including prior null responders, partial responders, and relapsers. Both boceprevir and telaprevir are approved for administration in combination with peginterferon and ribavirin only; neither is approved for monotherapy or for administration with ribavirin alone. (INCIVEK® (telaprevir) FDA-approved label, revised June 2012; VICTRELIS® (boceprevir) FDA-approved label, revised July 2012.)

The introduction of both boceprevir and telaprevir has increased the therapeutic options available to HCV-infected patients; however, both treatment regimens have certain disadvantages. A principle disadvantage is that the boceprevir and telaprevir regimens still require the use of peginterferon. Additional disadvantages are summarized below.

Boceprevir (used in combination with peginterferon α-2a and ribavirin) has a complicated dosing regimen, e.g., 800 mg (4×200 mg) three times daily (every 7 to 9 hours) with food. Moreover, late-stage clinical studies show that boceprevir used in combination with peginterferon and ribavirin results in a 66% SVR rate. (Manns et al., Liver Int'l (2012) 27-31.) Additionally, the boceprevir regimen must be administered for 48 weeks, which means that the treatment costs are quite expensive. Finally, use of boceprevir in combination with peginterferon and ribavirin is presently limited to those subjects infected with HCV genotype 1.

The telaprevir regimen (used in combination with peginterferon and ribavirin) requires a dosing regimen of 750 mg (2×375 mg) three times daily (7-9 hours apart) with food. An SVR rate of 79% was reported for patients receiving telaprevir in combination with peginterferon and ribavirin for 12 weeks. (Jacobson et al., New Engl. J. Med. (2011) 364: 2405-2416.) However, reports reveal that about half of the treated patients developed a skin rash or itching, and a small number of patients developed the severe Stevens-Johnson Syndrome, a life-threatening skin condition, in which case the regimen must be terminated. Finally, use of telaprevir in combination with peginterferon and ribavirin is presently limited to those subjects infected with HCV genotype 1. Although the treatment period is reduced for telaprevir as compared to that for boceprevir, the treatment costs for the two regimens are about the same.

Despite the additional options offered by the boceprevir and telaprevir regimens, these alternative treatments still have disadvantages and limitations. Further, genotype 1 patients who fail therapy with boceprevir and/or telaprevir in combination with peginterferon and ribavirin may develop undesirable NS3 protease inhibitor resistance (e.g., Pawlotsky, Hepatology (2011) 53(5): 1742-1751.) There is a need for improved treatment regimens that are more effective, safe, tolerable, shorter in duration, and which are associated with reduced rates of viral breakthrough and/or viral resistance. In particular, there is a need for interferon-free treatment regimens that are effective for treating CHC but result in reduced side-effects compared to treatment regimens involving interferon or peginterferon. There is also a need for interferon-free treatment regimens for patients suffering from CHC infection who are interferon-ineligible or interferon-intolerant. There is also a need to provide a therapy resulting in improved SVR compared to the outcome of treatment with peginterferon alone or in combination with ribavirin. In addition, there is a need to provide a therapy that reduces the time in which patients show evidence of complete viral suppression (negative HCV status) following the initiation of treatment in order to reduce side effects and costs.

Sofosbuvir (SOF), marketed under the trademark SOVALDI® (Gilead Sciences, Inc.), is a FDA-approved drug product approved for use as a component of a combination antiviral treatment regimen to treat chronic hepatitis C virus (HCV) infection. Sofosbuvir is a HCV nucleotide analog NS5B polymerase inhibitor indicated for the treatment of chronic hepatitis C(CHC) infection as a component of a combination antiviral treatment regimen.

Ledipasvir (LDV) is an investigational NS5A inhibitor currently in Phase 3 clinical trials for the treatment of chronic HCV infection.

Phase 3 clinical trials have been conducted to evaluate the efficacy, safety and tolerability of sofosbuvir 400 mg administered for 12 weeks with ribavirin with or without ledipasvir in treatment-naïve patients with chronic HCV GT 1 infection and prior null responder patients with chronic HCV GT 1 infection. The results of these trials, along with the results of in vitro studies, revealed several potential and hereto unknown advantages of HCV treatment regimens utilizing sofosbuvir in combination with ribavirin and ledipasvir. These results provide a basis for the disclosed and claimed method for treating HCV infection.

### SUMMARY

Disclosed herein is a method of treating a subject infected with hepatitis C virus (HCV), said method comprising administering to the subject for a time period an effective amount of sofosbuvir, an effective amount of ribavirin and an effective amount of ledipasvir. In one aspect, the method comprises administering to the subject an interferon-free treatment regimen comprising an effective amount of sofosbuvir, an effective amount of ribavirin and an effective amount of ledipasvir. In a particular aspect, the method is sufficient to produce an undetectable amount of HCV RNA in the subject for at least 12 weeks or for at least 24 weeks after the end of the time period. In another particular aspect of the invention, the patient is a treatment-naïve patient with chronic HCV GT 1 infection or a prior null responder, partial responder and relapser patient with chronic HCV GT 1 infection.

In one embodiment, the time period is from about 1 week to about 12 weeks. The time period may selected from 1 week, 2 weeks, 3 weeks, 4 weeks, 5 weeks, 6 weeks, 7 weeks, 8 weeks, 9 weeks, 10 weeks, 11 weeks and 12 weeks. In certain aspects of the invention the time period may be 4 weeks, 8 weeks or 12 weeks.

### BRIEF DESCRIPTION OF THE DRAWINGS

FIG. 1 shows baseline characteristics of patient populations in clinical trials: (1) treatment-naïve patients with chronic HCV GT 1 infection treated with sofosbuvir (SOF) and ribavirin (RBV); (2) prior null responder patients with chronic HCV GT 1 infection treated with SOF and RBV; (3) treatment-naïve patients with chronic HCV GT 1 infection treated with SOF, RBV and ledipasvir (LDV); and (4) prior null responder patients with chronic HCV GT 1 infection treated with SOF, RBV and LDV.

FIG. 2 shows the number of patients from the four patient populations, n/N (%), of FIG. 1 having less than the detectable amount of viral load (HCV RNA<LOD) after 1, 2, 4 and 12 weeks of treatment and reached SVR-4 and SVR-12 after having completed 12 weeks of treatment.

FIG. 3 shows the of HCV RNA (log 10 IU/mL) levels during 12 weeks of treatment and for up to 24 weeks after the end of treatment (EOT) for HCV GT1 treatment-naïve patients and HCV GT1 null responders receiving a combination of sofosbuvir (400 mg QD) and RBV (1000/1200 mg BID based on weight) for 12 weeks (FIG. 3A and FIG. 3B, respectively); and for HCV GT1 treatment-naïve patients and HCV GT1 null responders receiving a combination of sofosbuvir (400 mg QD), RBV (1000/1200 mg BID based on weight), and ledipasvir (90 mg QD) for 12 weeks (FIG. 3C and FIG. 3D, respectively).

FIG. 4 shows adverse events (AEs) for each of the four patient populations of FIG. 1.

FIG. 5 shows grade 3 and 4 lab abnormalities for each of the four patient populations of FIG. 1.

FIG. 6 shows the HCV RNA (log 10 IU/mL) levels during 3-day ledipasvir (LDV) treatment and for up to 4 days after the end of treatment (one week total) for HCV GT1a or 1b patients receiving 1 mg, 3 mg, 10 mg, 30 mg or 90 mg ledipasvir.

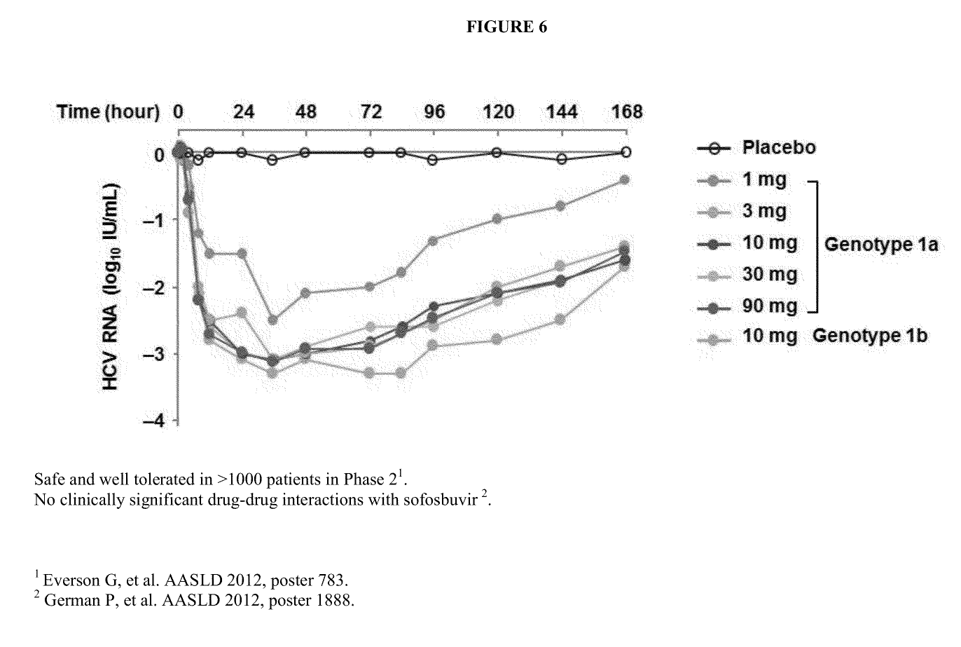

### DETAILED DESCRIPTION

The present disclosure is directed to methods of treating a subject infected with hepatitis C virus (HCV), said method comprising administering to the subject for a time period an effective amount of sofosbuvir, an effective amount of ribavirin and an effective amount of ledipasvir. In one aspect, the method comprises administering to the subject an interferon-free treatment regimen comprising an effective amount of sofosbuvir, an effective amount of ribavirin and an effective amount of ledipasvir. In a particular aspect, the method is sufficient to produce an undetectable amount of HCV RNA in the subject for at least 12 weeks after the end of the time period. In another particular aspect, the method is sufficient to produce an undetectable amount of HCV RNA in the subject for at least 24 weeks after the end of the time (EOT) period. In a further particular aspect of the invention, the patient is a treatment-naïve patient with chronic HCV GT 1 infection or a prior null responder, partial responder or relapser patient with chronic HCV GT 1 infection. In one aspect of the invention, the patient is a GT 1 null responder. The methods of the present invention provide safe and tolerable treatment regimens for these patient populations.

Sofosbuvir is a nucleotide analog prodrug, the 5′-triphosphate metabolite of which is a potent and selective inhibitor of HCV replication mediated by the HCV NS5B RNA dependent RNA polymerase (e.g., Sofia et al., J. Med. Chem. (2010) 53(19):7202-7218). In vivo, sofosbuvir is converted to 5′-mono-, di- and triphosphate metabolites (see ibid.). Further, sofosbuvir has demonstrated a low incidence of resistance and has been well-tolerated in clinical trials. Sofosbuvir (SOF) and previously known as or “GS-7977” or “PSI-7977”, is owned by Gilead Sciences, Inc.

Ledipasvir (LDV), also known as “GS-5885”, is owned by Gilead Sciences, Inc.

Ribavirin (RBV) is a guanosine analogue marketed under the trademark COPEGUS® and described in the Merck Index (12th Edition), monograph no. 8365. Ribavirin is indicated for the treatment of HCV in combination with PEG (see, e.g., Poynard et al., “Randomised Trial of Interferon Alpha2b Plus Ribavirin for 48 Weeks or for 24 Weeks Versus Interferon Alpha2b Plus Placebo for 48 Weeks for Treatment of Chronic Infection with Hepatitis C Virus,” Lancet (1998) 352(9138):1426-32; McHutchison et al., “Interferon Alfa-2b Alone or in Combination with Ribavirin as Initial Treatment for Chronic Hepatitis C,” N. Engl. J. Med. (1998) 339(21):1485-92). Ribavirin alone is not approved for the treatment of HCV infection. Ribavirin monotherapy has been reported to result in normalized alanine aminotransferase (ALT) levels transiently during the course of treatment, but ribavirin alone has little or no effect on HCV RNA levels during or after therapy and has not been shown to produce SVR.

### DEFINITIONS

Sofosbuvir (SOF), (sofosbuvir, formerly GS-7977 or PSI-7977) (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate, is represented by the following chemical structure:

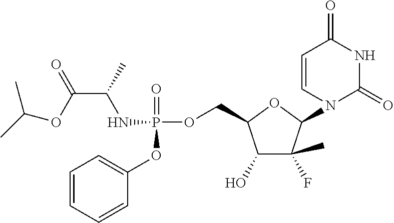 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

(see U.S. Pat. Nos. 7,964,580, 8,642,756, and 8,618,076; U.S. Patent Application Pub. Nos. 2010/0016251, 2010/0298257, 2011/0251152 and 2012/0107278).

Sofosbuvir may additionally be referred to as: (S)-isopropyl 2-((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphorylamino)propanoate; (S)-2-{[(1R,4R,5R)-5-(2,4-dioxo-3,4-dihydro-2H-pyrimidin-1-yl)-4-(R)-fluoro-3-hydroxy-4-methyl-tetrahydro-furan-2-ylmethoxy]-phenoxy-phosphorylamino}-propionic acid (S)-isopropyl ester; 2′-deoxy-2′-fluoro-2′-C-methyluridine 5′-Sp-[phenyl(isopropoxy-L-alaninyl)]phosphate; and L-alanine, N—[[P(S),2′R]-2′-deoxy-2′-fluoro-2′-methyl-P-phenyl-5′-uridylyl]-, 1-methylethyl ester. As referred to herein, the 5′-mono-, di- and triphosphate metabolites of sofosbuvir are represented by the following chemical structures:

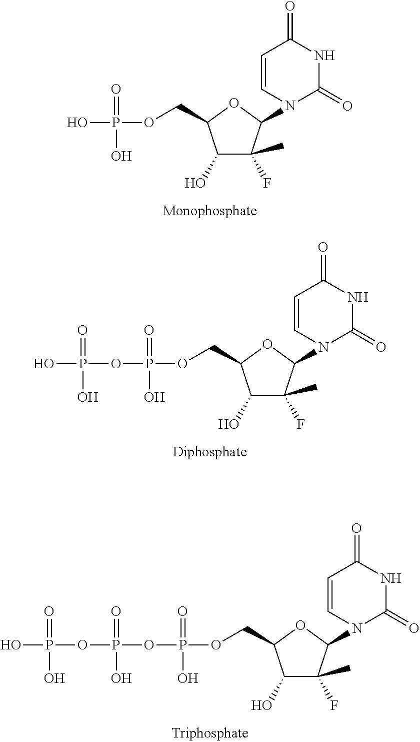 [SMILES: C[C@@]1(F)[C@H](O)[C@@H](COP(=O)(O)O)O[C@H]1n1ccc(=O)[nH]c1=O ; C[C@@]1(F)[C@H](O)[C@@H](COP(=O)(O)OP(=O)(O)O)O[C@H]1n1ccc(=O)[nH]c1=O ; C[C@@]1(F)[C@H](O)[C@@H](COP(=O)(O)OP(=O)(O)OP(=O)(O)O)O[C@H]1n1ccc(=O)[nH]c1=O]

Ribavirin, 1-β-d-ribofuranosyl-1H-1,2,4-triazole-3-carboxamide, is represented by the following chemical structure:

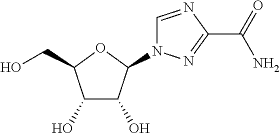 [SMILES: NC(=O)c1ncn([C@@H]2O[C@H](CO)[C@@H](O)[C@H]2O)n1]

(see, e.g., U.S. Pat. No. 4,530,901).

Ledipasvir (LDV), also known as GS-5885 and (1-{3-[6-(9,9-difluoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carbonyl}-2-methyl-propyl)-carbamic acid methyl ester, is an inhibitor of the HCV non-structural protein 5A (NS5A), and is represented by the following chemical structure:

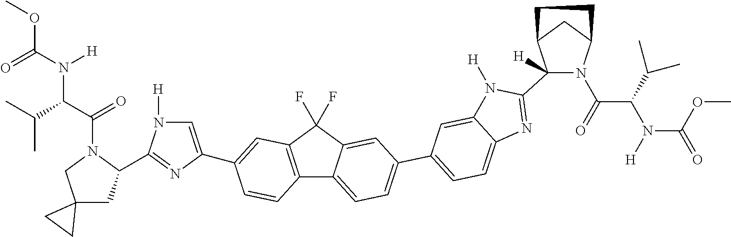

(see, e.g., U.S. Patent Application Pub. No. 20100310512 A1.)

“ALT” refers to alanine aminotransferase. Increased serum ALT levels can accompany hepatocellular injury or necrosis of striated muscle. In addition, release of ALT from the cytosol may occur secondary to cellular necrosis or as a result of cellular injury with membrane damage. An increased serum ALT level is one indication of hepatic damage.

“AST” refers to aspartate aminotransferase, an enzyme normally found in the blood in low levels. Increased serum AST may result from disease or damage to an organ, including the liver. An increased serum AST level is one indication of hepatic damage.

“Body mass index” (BMI) refers to the ratio of a subject's weight to the subject's height. BMI is expressed in units of kg/m2 and is calculated as follows:

$$BMI = \frac{weight ( pounds ) \times 703}{{( height in inches )}^{2}} or \frac{weight in kilograms}{{( height in meters )}^{2}}$$

“Effective amount” as used herein refers to an amount sufficient to reduce the symptoms of an HCV infection in a subject.

“GT” refers to genotype. There are several recognized HCV genotypes (1, 2, 3, 4, 5, 6, 7, 8, 9, 10 and 11), which are further categorized by different sub-types: 1 (1a, 1b, and 1c), 2 (2a, 2b, 2c), 3 (3a and 3b), 4 (4a, 4b, 4c, 4d, and 4e), 5 (5a), 6 (6a), 7 (7a and 7b), 8 (8a and 8b), 9 (9a), 10 (10a), and 11 (11a).

“IL28B” refers to interleukin 28B, which is one isoform of the interleukin-28 (IL28) cytokine There are several sub-types of the IL28B gene, which include CC, CT and TT genotypes.

“IU” refers to international unit, which is a measure of the amount of a substance based on its biological activity or effect.

“LOD” refers to the limit of detection. As used herein in reference to HCV RNA measurements, the LOD is about 15 IU/mL, as measured by TaqMan® HCV Test 2.0, available from Invitrogen, having a limit of detection of 15 IU/mL.

“Subject” as used herein refers to a human.

“Treatment-naïve subject” refers to a subject who has received no prior therapy for HCV (including interferon, sofosbuvir, ribavirin, ledipasvir or any other anti-HCV drug).

“Null responder” refers to a subject who has previously been treated with an interferon-based treatment regimen and did not achieve at least a 2 log 10 reduction in HCV RNA by week 12 of treatment.

“Partial responder” refers to a subject who has previously been treated with an interferon-based treatment regimen and achieved at least a 2 log 10 reduction in HCV RNA at week 12 but did not achieve an undetectable viral load at the end of treatment.

“Relapser” refers to a subject who has previously been treated with an interferon-based treatment regimen and achieved HCV RNA below the limit of detection (“LOD”) at the end of treatment but then relapsed back to having detectable HCV RNA levels post-treatment.

“RVR” refers to rapid virologic response (see, e.g., Poordad et al., Clin. Infect. Dis. (2008) 46:78-84).

A “sustained virologic response” (SVR) for a subject treated according to one of the methods described herein refers to the subject having HCV RNA less than the limit of detection (LOD, e.g., <about 15 IU/mL [as measured by TaqMan® HCV TTest 2.0, available from Invitrogen, Grand Island, N.Y., having a limit of detection of 15 IU/mL]) for a period of time post-treatment as measured in accordance with the assay methodology described herein.

“SVR-N” is the abbreviation for sustained virologic response N weeks after completion of one of the HCV treatment regimens disclosed herein. For example, SVR-4 is the abbreviation for sustained virologic response 4 weeks after completion of treatment.

“QD” means that a dose is administered once a day.

“BID” means that the dose is administered twice a day. The term “divided BID,” as used herein, means that a total dose amount is divided into two doses administered at different times during the course of a day. It is contemplated that the two doses into which a recited amount of a drug is “divided BID” may be equal or different.

“TID” means that a dose is administered three times a day.

“QID” means that a dose is administered four times a day.

### EMBODIMENTS

A first embodiment is directed to a method for treating a subject infected with hepatitis C virus, said method comprising administering to the subject for a time period an effective amount of sofosbuvir, an effective amount of ribavirin and an effective amount of ledipasvir.

In a first aspect of the first embodiment, the time period is selected from among about 1 week to about 12 weeks, about 2 weeks to about 12 weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, and about 11 weeks to about 12 weeks. In one subembodiment the time period is about 1 week to about 8 weeks, about 2 weeks to about 8 weeks, about 3 weeks to about 8 weeks, about 4 weeks to about 8 weeks, about 5 weeks to about 8 weeks, about 6 weeks to about 8 weeks and about 7 weeks to about 8 weeks. In a further subembodiment, the time period is about 1 week to about 4 weeks, about 2 weeks to about 4 weeks, and about 3 weeks to about 4 weeks. In a further subembodiment, the time period is selected from the group consisting of 1 week, 2 weeks, 3 weeks, 4 weeks, 5 weeks, 6 weeks, 7 weeks, 8 weeks, 9 weeks, 10 weeks, 11 weeks and 12 weeks. In one preferred subembodiment the time period is 12 weeks. In a further preferred subembodiment, the time period is 8 weeks. In another preferred embodiment, the time period is 4 weeks.

In a second aspect of the first embodiment, the method comprises administering to the subject an effective amount of sofosbuvir selected from among about 100 mg to about 800 mg per day, about 200 mg to about 800 mg per day, about 400 mg to about 800 mg per day, about 600 mg to about 800 mg per day, about 100 mg to about 600 mg per day, about 100 mg to about 400 mg per day, about 100 mg to about 200 mg per day, about 200 mg to about 600 mg per day, about 200 mg to about 400 mg per day, about 400 mg to about 600 mg per day, and about 400 mg per day. In one subembodiment, the method comprises administering to the subject about 400 mg per day of sofosbuvir. In another subembodiment, the method comprises administering sofosbuvir to the subject QD, BID, TID or QID. In one preferred subembodiment, the method comprises administering to the subject about 400 mg per day of sofosbuvir QD.

In a third aspect of the first embodiment, the method comprises administering to the subject an effective amount of ribavirin selected from about 100 mg to about 1400 mg per day, about 200 mg to about 1200 mg per day, about 400 mg to about 1200 mg per day, about 600 mg to about 1200 mg per day, about 800 mg to about 1200 mg per day, about 1000 mg to about 1200 mg per day, about 200 mg per day, about 400 mg per day, about 600 mg per day, about 800 mg per day, about 1000 mg per day, and about 1200 mg per day. In one subembodiment, the method comprises administering to the subject about 1000 mg to about 1200 mg per day of ribavirin. In another subembodiment, the method comprises administering to the subject about 1000 mg to about 1200 mg per day of ribavirin based on the subject's body weight. In another subembodiment, the method comprises administering to the subject about 1000 mg or about 1200 mg per day of ribavirin. In another subembodiment, the method comprises administering to the subject about 1000 mg or about 1200 mg per day of ribavirin based on the subject's body weight. In another subembodiment, the method comprises administering to the subject about 800 mg per day of ribavirin. In another subembodiment, the method comprises administering ribavirin to the subject QD, BID, TID, or QID. In another subembodiment, the method comprises administering ribavirin to the subject BID. In one preferred subembodiment, the method comprises administering to the subject about 1000 mg to about 1200 mg per day of ribavirin, divided BID. In another preferred subembodiment, the method comprises administering to the subject about 1000 mg or about 1200 mg per day of ribavirin, based on the subject's body weight, divided BID.

In a fourth aspect of the first embodiment, the method comprises administering to the subject an effective amount of ledipasvir selected from about 10 mg to about 100 mg per day, about 20 mg to about 100 mg per day, about 30 mg to about 100 mg per day, about 40 mg to about 100 mg per day, about 50 mg to about 100 mg per day, about 60 mg to about 100 mg per day, about 70 mg to about 100 mg per day, about 80 mg to about 100 mg per day, or about 90 mg to about 100 mg per day. In one subembodiment, the method comprises administering to the subject about 80 mg to about 100 mg per day of ledipasvir. In another subembodiment, the method comprises administering to the subject about 80 mg to about 100 mg per day of ledipasvir based on the subject's body weight. In another subembodiment, the method comprises administering to the subject about 90 mg per day of ledipasvir. In another subembodiment, the method comprises administering to the subject about 90 mg per day of ledipasvir base on the subject's body weight. In another subembodiment, the method comprises administering ledipasvir to the subject QD, BID, TID, or QID. In another subembodiment, the method comprises administering ledipasvir to the subject QD. In another subembodiment, the method comprises administering to the subject 90 mg of ledipasvir QD.

In a fourth aspect of the first embodiment, the method comprises administering to the subject about 100 mg to about 800 mg per day of sofosbuvir, about 100 mg to about 1400 mg per day of ribavirin, and about 80 mg to about 100 mg per day of ledipasvir. In one subembodiment, the method comprises administering to the subject about 400 mg per day of sofosbuvir, about 1000 mg to about 1200 mg per day of ribavirin, and about 90 mg per day of ledipasvir. In another subembodiment, the method comprises administering the effective amount of sofosbuvir QD, the effective amount of ribavirin divided into multiple doses (e.g., BID, TID, or QID) and the effective amount of ledipasvir QD. In one preferred subembodiment, the method comprises administering to the subject about 400 mg per day of sofosbuvir QD, about 1000 mg or about 1200 mg per day of ribavirin, based on the subject's body weight, divided BID, and about 90 mg per day of ledipasvir QD.

In a fifth aspect of the first embodiment, the subject has HCV RNA<LOD for at least 4 weeks after the end of the time period. In one subembodiment, the subject has HCV RNA<LOD for at least 8 weeks after the end of the time period. In another subembodiment, the subject has HCV RNA<LOD for at least 12 weeks after the end of the time period. In another subembodiment, the subject has HCV RNA<LOD for at least 24 weeks after the end of the time period. In a further subembodiment, the subject has HCV RNA<LOD for at least 48 weeks after the end of the time period.

In a sixth aspect of the first embodiment, the subject has less than about 15 IU/mL of HCV RNA for at least 4 weeks after the end of the time period. In one subembodiment, the subject has less than about 15 IU/mL of HCV RNA for at least 8 weeks after the end of the time period. In another subembodiment, the subject has less than about 15 IU/mL of HCV RNA for at least 12 weeks after the end of the time period. In another subembodiment, the subject has less than about 15 IU/mL of HCV RNA for at least 24 weeks after the end of the time period. In a further subembodiment, the subject has less than about 15 IU/mL of HCV RNA for at least 48 weeks after the end of the time period.

In a seventh aspect of the first embodiment, the subject is a treatment-naïve subject, a null responder subject, a partial responder subject or a relapser subject. In one subembodiment, the subject is treatment-naïve. In another subembodiment, the subject is a null responder. In a further subembodiment, the subject is a partial responder. In yet a further subembodiment, the subject is a relapser.

In an eighth aspect of the first embodiment, the HCV genotype of the subject is genotypes (GT) 1, 2, 3, 4, 5, 6, 7, 8, 9, 10 or 11, which are further categorized by different sub-types: 1 (1a, 1b, and 1c), 2 (2a, 2b, 2c), 3 (3a and 3b), 4 (4a, 4b, 4c, 4d, and 4e), 5 (5a), 6 (6a), 7 (7a and 7b), 8 (8a and 8b), 9 (9a), 10 (10a), and 11 (11a). In one subembodiment, the patient is GT 1.

A second embodiment of the invention is directed to a method for treating a subject infected with hepatitis C virus, said method comprising administering to the subject for a time period an effective amount of sofosbuvir, an effective amount of ribavirin, an effective amount of ledipasvir, and at least one additional therapeutic agent.

With respect to the foregoing embodiments, it is contemplated that administration of sofosbuvir, ribavirin and ledipasvir may be concurrent, alternate or any variation thereof, meaning that when the effective amounts of sofosbuvir, ribavirin and ledipasvir are administered during the same time period, the specific order of administration on a daily basis can be: sofosbuvir followed by ribavirin and/or ledipasvir, sofosbuvir, ribavirin and ledipasvir together; ribavirin followed by sofosbuvir and/or ledipasvir; ledipasvir followed by sofosbuvir and/or ribavirin, or any variation thereof. Additionally, it is contemplated that sofosbuvir, ribavirin and ledipasvir dosage frequencies may differ. As one non-limiting example, sofosbuvir and ledipasvir may be administered QD while ribavirin is administered BID.

Also with respect to the foregoing embodiments, sofosbuvir may be administered orally in capsule or tablet form, or in any other suitable unit dosage form, in association with the oral (e.g., capsule or tablet form) administration of ribavirin and oral (e.g., capsule or tablet form) of ledipasvir. Other routes of administration of both medicaments, as they become available, are contemplated, such as by nasal spray, by a buccal or sublingual administration dosage form, transdermally, by suppository, by sustained release dosage form, etc.

Further with respect to the foregoing embodiments, it is contemplated that the 5′-mono-, di- and/or triphosphate metabolites of sofosbuvir may be provided to a subject by administering sofosbuvir to the subject, as well as by other means not involving the administration of sofosbuvir. For example, it is contemplated that the 5′-mono-, di- and/or triphosphate metabolites of sofosbuvir may be provided to a subject by administering compounds other than sofosbuvir that are converted to the disclosed 5′-mono-, di- and/or triphosphate metabolites in vivo.

The methods disclosed herein may be part of an interferon-free method of treatment. However, should it be desired, the method of treatment may further comprise interferon.

### EXAMPLES

The disclosure is further illustrated in the following non-limiting studies. These studies are set forth to aid in the understanding of the disclosure but are not intended, and should not be construed, to limit its scope in any way. The descriptions of the studies do not include detailed descriptions of conventional methods that are well-known to those of ordinary skill in the art.

In the following examples and throughout this disclosure, abbreviations as used herein have respective meanings as follows:

| | |
|---|---|
| AE(s) | Adverse Event(s) |
| ALT | Alanine Aminotransferase |
| AST | Aspartate Aminotransferase |
| BL | Baseline |
| BID | Dose Administered Twice a Day |
| BMI | Body Mass Index |
| CC, CT, or | Genotypes of IL28B Gene |
| TT | |
| CHC | Chronic Hepatitis C |
| dL | Deciliter |
| EOT | End of Treatment |
| FDA | Federal Drug Administration |
| g | Grams |
| GT | Genotype |
| HCC | Hepatocellular Carcinoma |
| HCV | Hepatitis C Virus |
| HIV | Human Immunodeficiency Virus |
| IL28B | Interleukin 28B |
| IU | International Unit |
| kD | Kilodalton |
| kg | Kilogram |
| LDV | Ledipasvir (GS-5885) |
| LOD | Limit of Detection |
| M | Molar |
| mg | Milligram |
| mL | Milliliter |
| n | Number of Subjects |
| N | Population Size |
| PCR | Polymerase Chain Reaction |
| PEG | Pegylated Interferon |
| QD | Dose Administered Once a Day |
| QID | Dose administered Four Times a Day |
| RBV | Ribavirin |
| RNA | Ribonucleic Acid |
| SAE(s) | Severe Adverse Event(s) |
| SOC | Standard of Care |
| SOF | Sofosbuvir (GS-7977, formerly PSI-7977) |
| SVR | Sustained Virologic Response |
| SVR-N | Sustained Virologic Response N-Weeks |
| TID | Dose Administered Three Times a Day |
| ULN | Upper Limit of Detection |

### Example 1

### Sofosbuvir and Ribavirin Administered to Treatment-Naïve Subjects with Chronic HCV GT 1

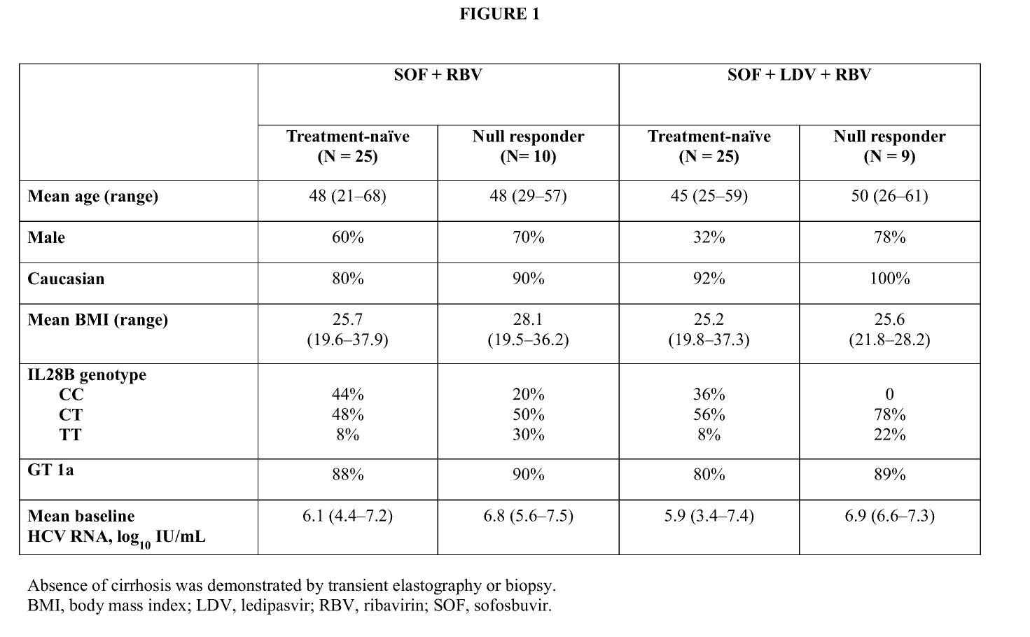

In this open-label study, 25 treatment-naïve HCV GT1-infected subjects were administered a combination of sofosbuvir and ribavirin for 12 weeks. The baseline characteristics of the patients are shown in FIG. 1. FIG. 1 shows the mean age of this population to be 48 with a range from 21 to 68. The population was 60% male and 80% caucasian. The mean BMI was 25.7 with a range from 19.6 to 37.9. The IL28B genotype was CC 44%, CT 48% and TT 8%. The population was 88% GT 1a. The mean baseline HCV RNA, log 10 IU/mL of the population was 6.1, with a range of 4.4 to 7.2.

Subjects received a total daily oral dose of 400 mg of sofosbuvir QD and a total daily oral dose of 1000 mg (weight<75 kg) or 1200 mg (weight≧75 kg) of ribavirin, divided BID, for 12 weeks. Treatment was discontinued after 12 weeks. Subjects' HCV RNA levels were measured at the initial screening visit, baseline (day 1 of treatment), weeks 1, 2, 3, 4 and then every 4 weeks through the end of treatment (12 weeks total).

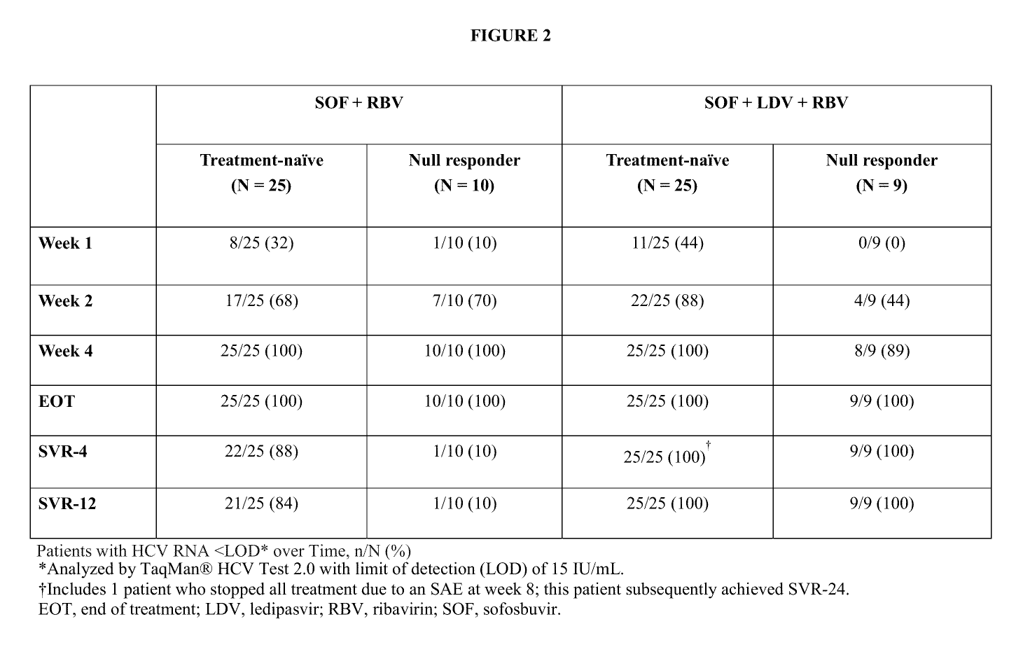

FIG. 2 shows that after week 4, 100% of the subjects had less than the limit of detection (LOD) of HCV RNA as analyzed by the TaqMan HCV Test 2.0, available from Invitrogen of Grand Island, N.Y., which has a LOD of 15 IU/mL. FIG. 2 also shows that at the end of the 12 week treatment, 100% of the subjects still had less than the LOD of HCV RNA.

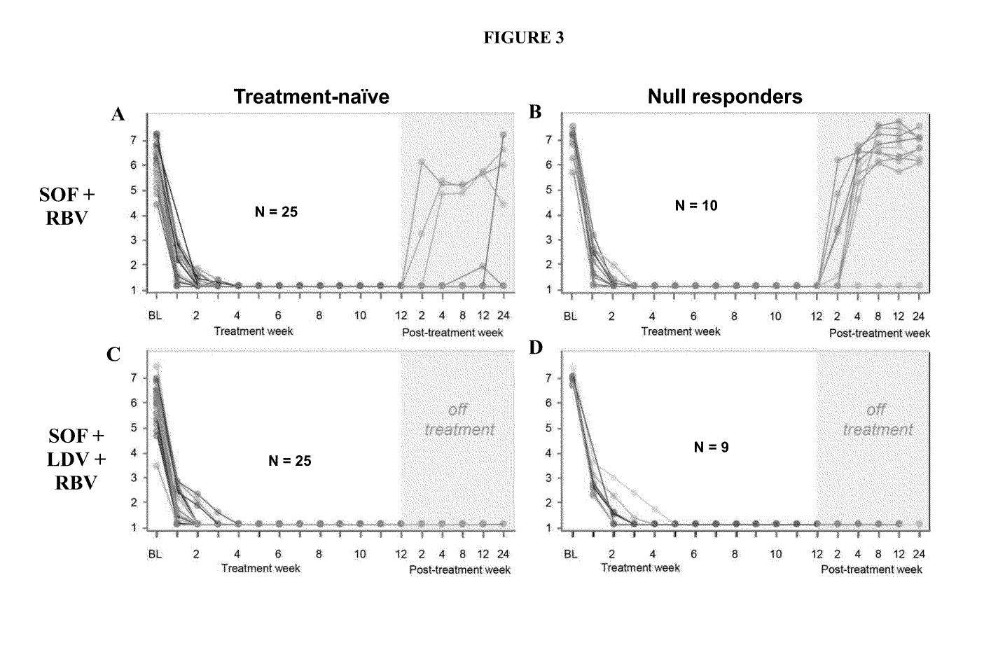

After 12 weeks of treatment, patients were monitored for evidence of HCV infection as determined by HCV RNA>LOD (i.e., >about 15 IU/mL). As shown in FIG. 2, 21 of 25 subjects, i.e., 84% reached SVR-12. FIG. 2 also shows that 22 of 25 subjects, i.e., 88%, reached SVR-4. FIG. 3A, top left panel, is the graph showing the data provided in the table of FIG. 2. In FIG. 3A, it is seen that the study continued 24 weeks.

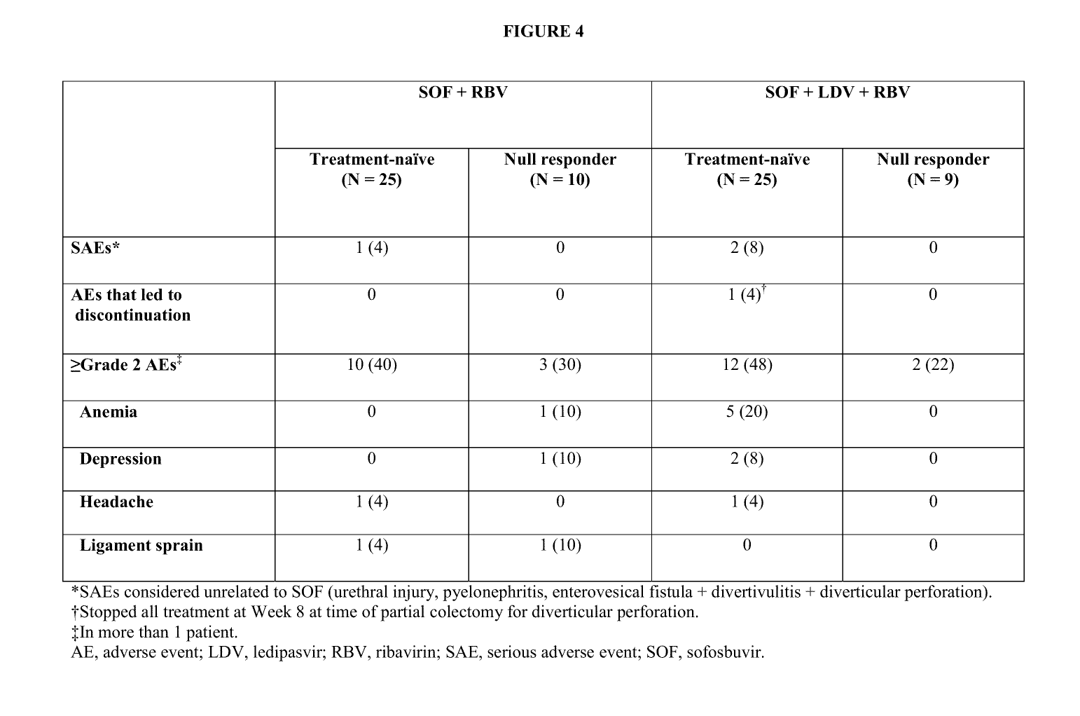

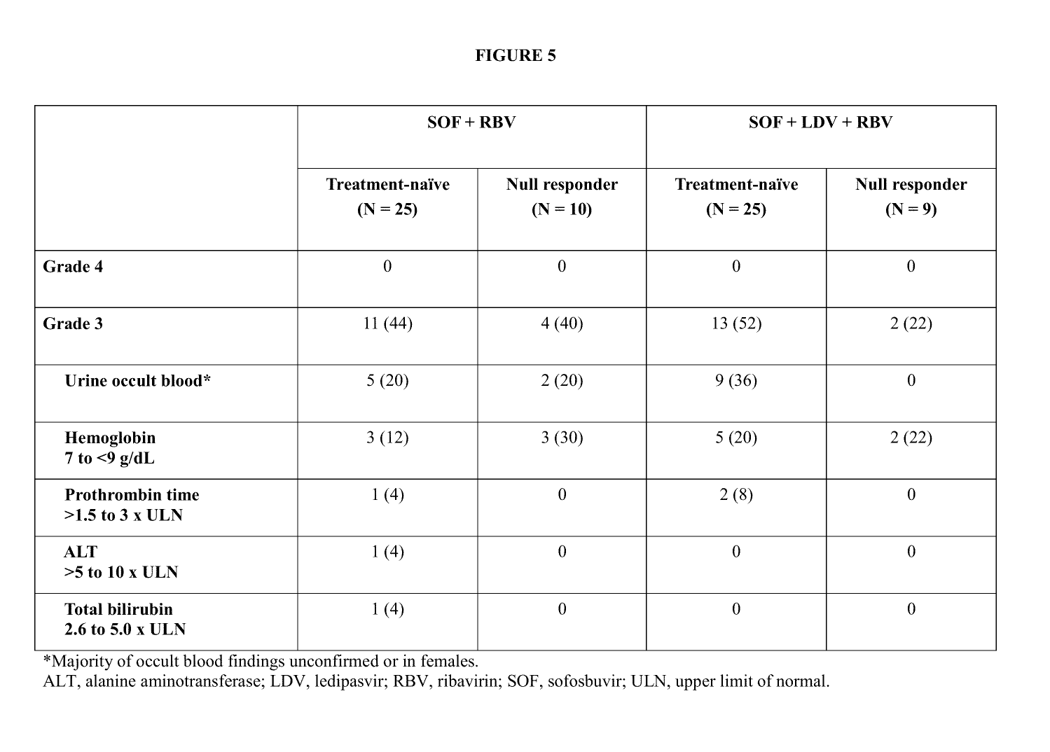

Adverse events during the study are shown in FIGS. 4 and 5.

HCV genotype and subtype can be determined using the Siemens VERSANT® HCV Genotype LiPA 2.0 Assay.

IL28B genotype can be determined by polymerase chain reaction (PCR) amplification of the SNP, rs12979860, with sequence specific forward and reverse primers and allele specific fluorescently labeled TaqMan® MGB probes.

### Example 2

### Sofosbuvir and Ribavirin Administered to Null Responder Subjects with Chronic HCV GT 1

In this open-label study, 10 null responder HCV GT1-infected subjects were administered a combination of sofosbuvir and ribavirin for 12 weeks. The baseline characteristics of the patients are shown in FIG. 1. FIG. 1 shows the mean age of this population to be 48 with a range from 29 to 57. The population was 70% male and 90% caucasian. The mean BMI was 28.1 with a range from 19.5 to 36.2. The IL28B genotype was CC 20%, CT 50% and TT 30%. The population was 90% GT 1a. The mean baseline HCV RNA level (log 10 IU/mL) of the population was 6.8, with a range of 5.6 to 7.5.

Subjects received a total daily oral dose of 400 mg of sofosbuvir QD and a total daily oral dose of 1000 mg (weight<75 kg) or 1200 mg (weight≧75 kg) of ribavirin, divided BID, for 12 weeks. Subjects' HCV RNA levels were measured at the initial screening visit, baseline (day 1 of treatment), weeks 1, 2, 3, 4 and then every 4 weeks through the end of treatment (12 weeks total). Treatment was discontinued after 12 weeks.

FIG. 2 shows that after week 4, 100% of the subjects had less than the limit of detection (LOD) of HCV RNA as analyzed by the TaqMan® HCV Test 2.0, available from Invitrogen of Grand Island, N.Y., which has a LOD of 15 IU/mL. FIG. 2 also shows that at the end of the 12 week treatment, 100% of the subjects still had less than the LOD of HCV RNA.

After 12 weeks of treatment, patients were monitored for evidence of HCV infection as determined by HCV RNA>LOD (i.e., >about 15 IU/mL). As shown in FIG. 2, 1 of 10 subjects, i.e., only 10% reached SVR-12. FIG. 2 also shows that 1 of 10 subjects, i.e., only 10%, reached SVR-4. FIG. 3B, top right panel, is the graph showing the data provided in the table of FIG. 2. In FIG. 3B, it is seen that the study continued 24. FIG. 3B also shows that although null responders did achieve RVR, 90% relapsed quickly at about 2 weeks after treatment was discontinued.

Adverse events during the study are shown in FIGS. 4 and 5.

HCV genotype and subtype can be determined using the Siemens VERSANT® HCV Genotype LiPA 2.0 Assay.

IL28B genotype can be determined by polymerase chain reaction (PCR) amplification of the SNP, rs12979860, with sequence specific forward and reverse primers and allele specific fluorescently labeled TaqMan® MGB probes.

### Example 3

### Sofosbuvir, Ribavirin and Ledipasvir Administered to Treatment-Naïve Subjects with Chronic HCV GT 1

In this open-label study, 25 treatment-naïve HCV GT1-infected subjects were administered a combination of sofosbuvir, ribavirin and ledipasvir for 12 weeks. The baseline characteristics of the patients are shown in FIG. 1. FIG. 1 shows the mean age of this population to be 45 with a range from 25 to 59. The population was 32% male and 92% caucasian. The mean BMI was 25.2 with a range from 19.8 to 37.3. The IL28B genotype was CC 36%, CT 56% and TT 8%. The population was 80% GT 1a. The mean baseline HCV RNA, log 10 IU/mL of the population was 5.9, with a range of 3.4 to 7.4.

Subjects received a total daily oral dose of 400 mg of sofosbuvir QD and 90 mg of ledipasvir QD along with a total daily oral dose of 1000 mg (weight<75 kg) or 1200 mg (weight≧75 kg) of ribavirin, divided BID, for 12 weeks. Subjects' HCV RNA levels were measured at the initial screening visit, baseline (day 1 of treatment), weeks 1, 2, 3, 4 and then every 4 weeks through the end of treatment (12 weeks total). Treatment was discontinued after 12 weeks.

FIG. 2 shows that after week 4, 100% of the subjects had less than the limit of detection (LOD) of HCV RNA as analyzed by the TaqMan® HCV Test 2.0, available from Invitrogen of Grand Island, N.Y., which has a LOD of 15 IU/mL. FIG. 2 also shows that at the end of the 12 week treatment, 100% of the subjects still had less than the LOD of HCV RNA.

After 12 weeks of treatment, patients were monitored for evidence of HCV infection as determined by HCV RNA>LOD (i.e., >about 15 IU/mL). As shown in FIG. 2, 100% of the subjects surprisingly reached SVR-12 compared to 84% of the subjects in the treatment-naïve group receiving only sofosbuvir and RBV. FIG. 2 also shows that 100% of the subjects surprisingly reached SVR-4, as compared to 88% of the subjects in the treatment-naïve group receiving only sofosbuvir and RBV. FIG. 3C, bottom left panel, is the graph showing the data provided in the table of FIG. 2. In FIG. 3C, it is seen that the study continued for at least some patients 24 weeks after treatment was discontinued. As shown in FIG. 2, treatment for one patient represented with a “f” was discontinued after 8 weeks. This patient achieved SVR-24.

Adverse events during the study are shown in FIGS. 4 and 5. No additional safety or tolerability issues were observed in the treatment-naïve subjects receiving sofosbuvir, RBV and ledipasvir, as compared to those receiving only sofosbuvir and RBV.

HCV genotype and subtype can be determined using the Siemens VERSANT® HCV Genotype LiPA 2.0 Assay.

IL28B genotype can be determined by polymerase chain reaction (PCR) amplification of the SNP, rs12979860, with sequence specific forward and reverse primers and allele specific fluorescently labeled TaqMan® MGB probes.

### Example 4

### Sofosbuvir, Ribavirin and Ledipasvir Administered to Null Responder Subjects with Chronic HCV GT 1

In this open-label study, 9 null responder HCV GT1-infected subjects were administered a combination of sofosbuvir, ribavirin and ledipasvir for 12 weeks. The baseline characteristics of the patients are shown in FIG. 1. FIG. 1 shows the mean age of this population to be 50 with a range from 26 to 61. The population was 78% male and 100% caucasian. The mean BMI was 25.6 with a range from 21.8 to 28.2. The IL28B genotype was CC 0%, CT 78% and TT 22%. The population was 89% GT 1a. The mean baseline HCV RNA, log 10 IU/mL of the population was 6.9, with a range of 6.6 to 7.3.

Subjects received a total daily oral dose of 400 mg of sofosbuvir QD and 90 mg of ledipasvir QD along with a total daily oral dose of 1000 mg (weight<75 kg) or 1200 mg (weight≧75 kg) of ribavirin, divided BID, for 12 weeks. Subjects' HCV RNA levels were measured at the initial screening visit, baseline (day 1 of treatment), weeks 1, 2, 3, 4 and then every 4 weeks through the end of treatment (12 weeks total). Treatment was discontinued after 12 weeks.

FIG. 2 shows that after week 4, 8 of 9 subjects, i.e., 89%, had less than the limit of detection (LOD) of HCV RNA as analyzed by the TaqMan® HCV Test 2.0, available from Invitrogen of Grand Island, N.Y., which has a LOD of 15 IU/mL. FIG. 2 also shows that at the end of the 12 week treatment, 100% of the subjects had less than the LOD of HCV RNA.

After 12 weeks of treatment, patients were monitored for evidence of HCV infection as determined by HCV RNA>LOD (i.e., >about 15 IU/mL). As shown in FIG. 2, remarkably, 100% of the subjects reached SVR-12, as compared to 10% of the null responder subjects that received only sofosbuvir and RBV. FIG. 2 also shows that remarkably 100% of the subjects reached SVR-4, as compared to 10% of the null responder subjects that received only sofosbuvir and RBV. FIG. 3D, bottom right panel, is the graph showing the data provided in the table of FIG. 2.

Adverse events during the study are shown in FIGS. 4 and 5. No additional safety or tolerability issues were observed in the null responder subjects receiving sofosbuvir, RBV and ledipasvir, as compared to those receiving only sofosbuvir and RBV.

HCV genotype and subtype can be determined using the Siemens VERSANT® HCV Genotype LiPA 2.0 Assay.

IL28B genotype can be determined by polymerase chain reaction (PCR) amplification of the SNP, rs12979860, with sequence specific forward and reverse primers and allele specific fluorescently labeled TaqMan® MGB probes.

1. A method for treating a subject infected with hepatitis C virus, said method comprising administering to the subject for a time period an effective amount of sofosbuvir or the 5′-mono-, di- and/or triphosphate metabolite of sofosbuvir, an effective amount of ribavirin and an effective amount of ledipasvir.

2. (canceled)

3. The method according to claim 1, wherein the time period is selected from among: about 1 week to about 12 weeks, about 2 weeks to about 12 weeks, about 3 weeks to about 12 weeks, about 4 weeks to about 12 weeks, about 5 weeks to about 12 weeks, about 6 weeks to about 12 weeks, about 7 weeks to about 12 weeks, about 8 weeks to about 12 weeks, about 9 weeks to about 12 weeks, about 10 weeks to about 12 weeks, and about 11 weeks to about 12 weeks.

4. The method according to claim 3, wherein the time period is about 12 weeks.

5. The method according to claim 3, wherein the time period is about 8 weeks.

6. The method accordingly to claim 3, wherein the time period is about 4 weeks.

7. The method according to claim 1, wherein the effective amount of sofosbuvir is selected from among about 100 mg to about 800 mg per day, about 200 mg to about 800 mg per day, about 400 mg to about 800 mg per day, about 600 mg to about 800 mg per day, about 100 mg to about 600 mg per day, about 100 mg to about 400 mg per day, about 100 mg to about 200 mg per day, about 200 mg to about 600 mg per day, about 200 mg to about 400 mg per day, about 400 mg to about 600 mg per day, and about 400 mg per day.

8. (canceled)

9. The method according to claim 7, wherein the effective amount of sofosbuvir is 400 mg per day.

10. The method according to claim 1, wherein the 5′-mono-, di- and/or triphosphate metabolite of sofosbuvir is provided by administering 400 mg per day of sofosbuvir to the subject.

11. The method according to claim 1, wherein the effective amount of ribavirin is selected from among about 100 mg to about 1400 mg per day, about 200 mg to about 1200 mg per day, about 400 mg to about 1200 mg per day, about 600 mg to about 1200 mg per day, about 800 mg to about 1200 mg per day, about 1000 mg to about 1200 mg per day, about 200 mg per day, about 400 mg per day, about 600 mg per day, about 800 mg per day, about 1000 mg per day, and about 1200 mg per day.

12. The method according to claim 11, wherein the effective amount of ribavirin is about 1000 mg to about 1200 mg based on the body weight of the subject.

13. The method according to claim 1, wherein the effective amount of ledipasvir is selected from among about 10 to about 100 mg per day, about 20 to about 100 mg per day, about 30 to about 100 mg per day, about 40 to about 100 mg per day, about 50 to about 100 mg per day, about 60 to about 100 mg per day, about 70 to about 100 mg per day, about 80 to about 100 mg per day, or about 90 to about 100 mg per day.

14. The method accordingly to claim 13, wherein the effective amount of ledipasvir is 90 mg per day.

15. The method according to claim 1, wherein the effective amount of sofosbuvir is about 400 mg per day, the effective amount of ribavirin is about 1000 mg to about 1200 mg per day, and the effective amount of ledipasvir is about 90 mg per day.

16. The method according to claim 1, wherein the subject has less than about 15 IU/mL of HCV RNA for at least 12 weeks after the end of the time period.

17. The method according to claim 1, wherein the subject has less than about 15 IU/mL of HCV RNA for at least 24 weeks after the end of the time period.

18. The method according to claim 1, wherein the subject is a treatment-naïve subject.

19. The method according to claim 1, wherein the subject is selected from the group consisting of a treatment-naïve subject, a null responder subject, a partial responder subject and a relapser subject.

20. The method according to claim 19, wherein the subject is a null responder subject.

21. The method according to claim 1, wherein the subject is infected with HCV genotype 1.

22. The method according to claim 1, further comprising administering to the subject at least one additional antiviral agent during the time period.

23. The method according to claim 1, wherein the subject is human.
